(19) United States
(12) Patent Application Publication
(10) Pub. No.: US 2023/0404765 A1
(43) Pub. Date: Dec. 21, 2023

(54) A SYSTEM AND METHOD FOR OSTEOARTHRITIS TREATMENT

(71) Applicant: UNIVERSITY OF CONNECTICUT, FARMINGTON, CT (US)

(72) Inventors: Thanh Duc Nguyen, South Windsor, CT (US); Thi Tra Nguyen, Storrs, CT (US)

(21) Appl. No.: 18/251,984

(22) PCT Filed: Oct. 7, 2021
(86) PCT No.: PCT/US2021/053887

§ 371 Date: May 5, 2023

Related U.S. Application Data
(60) Provisional application No. 63/122,155, filed on Dec. 7, 2020.

(51) Int. Cl. A61F 2/38; A61L 27/26; A61L 27/36

(52) U.S. Cl. CPC A61F 2/38; A61L 27/26; A61L 27/3654; A61F 2002/2864

(57) ABSTRACT

Methods and systems of osteoarthritis treatment. One method includes providing a cartilage hydrogel, the cartilage hydrogel including piezoelectric nano-fibers of Poly-L-lactide (PLLA). The method also includes injecting the cartilage hydrogel into a cartilage defect. The method also includes applying an ultrasonic treatment to the cartilage defect. The method also includes, in response to applying the ultrasonic treatment to the cartilage defect, converting a mechanical impact of the ultrasonic treatment into an electrical charge from the piezoelectric nano-fibers of PLLA and providing, in response to the electrical charge from the piezoelectric nano-fibers of PLLA, chondrogenesis differentiation for cartilage regeneration for the cartilage defect.

### CROSS-REFERENCE TO RELATED APPLICATIONS

This application is a non-provisional of and claims the benefit of U.S. Provisional Patent Application No. 63/122,155, filed on Dec. 7, 2020, the contents of which are incorporated herein by reference.

### STATEMENT REGARDING FEDERALLY SPONSORED RESEARCH OR DEVELOPMENT

This invention was made with government support under R21AR074645 awarded by National Institutes of Health. The government has certain rights in the invention.

### BACKGROUND

Millions of American suffer from osteoarthritis, and current medicines including analgesics and anti-inflammatory drugs only alleviate the symptoms but do not completely cure the disease. The golden treatment so far has been to use replacement auto-grafts and allo-grafts. These grafts however struggle with problems of donor site morbidity, immune-rejection, infection, and, especially, limit of tissue supply. Engineered cartilage grafts, constructed by seeding stem/chondrogenic cells onto biomaterial scaffolds along with growth factors, have emerged as a compelling alternative tissue source. Despite many encouraging results, clinical use of the engineered cartilage grafts is still limited due to the heavy dependence on toxic growth factors to induce chondrogenesis.

The concept of applying tissue engineering in cartilage regeneration has been limited due to the toxicity of growth factors, the inefficiency of cell differentiation, and invasive surgical processes to implant the grafts.

### SUMMARY

As an electrical signal has a significant effect on promoting tissue growth and is inherent in living organisms, electrical stimulation (ES) offers a natural and biocompatible approach for inducing cartilage regeneration. Piezoelectric materials with an exciting ability to convert mechanical deformation into electricity, appear to be an appealing platform to create self-powered electrical stimulators, which may either harvest mechanical joint-force or be externally stimulated by ultrasound to generate useful ES for cartilage growth. A novel biodegradable piezoelectric polymer, made of Poly-L-lactide (PLLA), is a well-known biocompatible material used for bone scaffolds, surgical sutures, and drug-delivery devices.

The present disclosure describes an injectable piezoelectric hydrogel for treatment of osteoarthritis (defect treatment in osteoarthritic knee). More particularly, an injectable piezoelectric collagen-based hydrogel, containing piezoelectric nano-fibers of PLLA, is described herein to enhance cartilage regeneration under ultrasound stimulus. The gel may also include stem cells to increase the chondrogenic differentiation. Through a minimally-invasive arthroscopic procedure, the hybrid hydrogel solution is injected into a cartilage defect and spontaneously cured under body temperature to form a cartilage graft in situ. The piezoelectric hydrogel is stimulated by ultrasound to generate useful surface charge, which may recruit stem cells and promotes chondrogenesis. This effect may even be enhanced by seeding mesenchymal stem cells (for example, adipose-derived stem cells, bone marrow stem cells, and the like) or other types of stem cells in the hydrogel.

The present disclosure provides (1) a minimally invasive treatment for patients as the piezoelectric hydrogel may be precisely placed into a defect through arthroscopic procedure or even injectable through a traditional needle; (2) a remote control and non-invasive ultrasonic treatment that is used for cartilage regeneration without additional growth factors; and (3) a piezoelectric hydrogel that is biocompatible and biodegradable, therefore, safe and will be circulated out of body after it has done its job. The hydrogel may also receive the joint loads from joint-bending to produce the surface charge. The present disclosure describes a combination of many novel concepts including piezoelectric nanofibers, non-invasive activation of piezo-charge, and injectable hydrogels to create a unique cartilage hydrogel that may self-generate electrical stimulation for cartilage regeneration, making this method highly innovative.

1. Hydrogel scaffold+short piezoelectric nanofibers of PLLA (may add adipose-derived stem cells (ADSCs)).

2. Apply mechanical stimulation (ultrasound or joint force itself), promotes generation of electricity from nanofibers, cell recruitment, and healing via chondrogenic differentiation of cells.

This disclosure provides a novel piezoelectric hydrogel for cartilage regeneration that employs a collagen hydrogel, which may contain adipose-derived stem cells (ADSC), and piezoelectric nanofibers of poly-L-lactide acid (PLLA) or similar piezoelectric materials. Under ultrasound stimulation, the piezoelectric hydrogel converts mechanical impact to electrical charge which is a useful factor for cell recruitment, chondrogenesis, and cartilage healing.

Additionally, this disclosure provides (1) a minimally invasive treatment for patients as the piezoelectric hydrogel can be precisely placed into the defects through arthroscopic procedure; (2) a remote control and non-invasive ultrasonic or joint-exercise treatment which is used for cartilage regeneration without additional growth factors; (3) the piezoelectric hydrogel is biocompatible and biodegradable, therefore, it is safe and will be circulated out of body after it has done its job. A combination of many novel concepts including piezoelectric nanofibers injectable hydrogels and with or without stem cells create a unique cartilage hydrogel which can self-generate electrical stimulation for cartilage regeneration make this method highly innovative.

In the treatment described herein, a less painful and minimally invasive method is provided because the hydrogel may be transferred to the defect through a needle. Therefore, patients may save time, money, and reduce risk by using the simple injection instead of going through multiple and complex steps of surgeries. The hydrogel creates charge under ultrasound treatment to signal stem cells to differentiate to cartilage without the need for growth factors.

Accordingly, embodiments described herein provide methods and systems of osteoarthritis treatment. One embodiment provides a method of osteoarthritis treatment. The method includes providing a cartilage hydrogel, the cartilage hydrogel including piezoelectric nano-fibers of Poly-L-lactide (PLLA). The method also includes injecting the cartilage hydrogel into a cartilage defect. The method also includes applying an ultrasonic treatment to the cartilage defect. The method also includes, in response to applying the ultrasonic treatment to the cartilage defect, converting a mechanical impact of the ultrasonic treatment into an electrical charge from the piezoelectric nano-fibers of PLLA and providing, in response to the electrical charge from the piezoelectric nano-fibers of PLLA, chondrogenesis differentiation for cartilage regeneration for the cartilage defect.

Another embodiment provides a system of osteoarthritis treatment. The system includes a cartilage hydrogel. The cartilage hydrogel includes piezoelectric nano-fibers of Poly-L-lactide (PLLA), wherein the cartilage hydrogel is injected into a cartilage defect, and wherein the cartilage hydrogel is configured to receive an ultrasonic treatment at the cartilage defect. In response to the ultrasonic treatment, the piezoelectric nano-fibers of PLLA converts a mechanical impact of the ultrasonic treatment into an electrical charge, wherein the electrical charge triggers a chondrogenesis differentiation for cartilage regeneration for the cartilage defect.

Other aspects of the embodiments will become apparent by consideration of the detailed description and accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an injectable piezoelectric hydrogel to treat a cartilage defect in an osteoarthritis knee.

FIG. 2 illustrates fabrication of piezoelectric nanofibers.

FIG. 3 is a graph illustrating the voltage output of piezoelectric PLLA fibers versus non-piezoelectric control fibers.

FIG. 4 is a graph confirming piezoelectricity of cut PLLA fibers by Rhodamine B dye.

FIGS. 5A-5D are graphs illustrating results of a frequency sweep relating to the injectable ability of PLLA fibers hydrogel.

FIGS. 6A-6B illustrate biocompatibility of piezoelectricity hydrogel with ADSC cells.

FIGS. 7A-7D are graphs illustrating the ability to induce chonodrogenesis of fabricated hydrogel in growth media.

FIGS. 8A-8D are graphs illustrating the ability to promote ADSC cells to differentiate to chondrocyte like cells of fabricated hydrogel in chondrogenesis media.

FIGS. 9A-9B are graphs illustrating the voltage output of three sensors made from different materials under ultrasound stimulation in rabbit's knee cadaver defect.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the embodiments described herein. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not. Further, it should further be noted that the terms “first,” “second,” and the like herein do not denote any order, quantity, or relative importance, but rather are used to distinguish one element from another.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. All ranges disclosed herein are inclusive of the endpoints, and the endpoints are independently combinable with each other. Each range disclosed herein constitutes a disclosure of any point or sub-range lying within the disclosed range.

As used herein, the terms “providing”, “administering,” and “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, “treat,” “treating” and the like mean a slowing, stopping or reversing of progression of a disease or disorder when provided a composition described herein to an appropriate control subject. The terms also mean a reversing of the progression of such a disease or disorder to a point of eliminating or greatly reducing the cell proliferation. As such, “treating” means an application or administration of the compositions described herein to a subject, where the subject has a disease or a symptom of a disease, where the purpose is to cure, heal, alleviate, relieve, alter, remedy, ameliorate, improve or affect the disease or symptoms of the disease.

All documents cited herein and the following listed documents that are attached hereto for submission, all referenced publications cited therein, and the descriptions and information contained in these documents are expressly incorporated herein in their entirety to the same extent as if each document or cited publication was individually and expressly incorporated herein.

While the embodiments have been described with reference to preferred embodiments, it will be understood by those skilled in the art that various changes may be made and equivalents may be substituted for the elements thereof without departing from the scope of the embodiments. In addition, many modifications may be made to adapt the teaching of the embodiments described herein to particular use, application, manufacturing conditions, use conditions, composition, medium, size, and/or materials without departing from the essential scope and spirit of the embodiments described herein.

While various aspects and embodiments have been disclosed herein, other aspects and embodiments will be apparent to those skilled in the art. The various aspects and embodiments disclosed herein are for purposes of illustration and are not intended to be limiting of the true scope of the embodiments disclosed herein. It is also to be understood that the terminology used herein is for the purpose of describing particular embodiments only, and is not intended to be limiting. Since many modifications, variations, and changes in detail can be made to the described examples, it is intended that all matters in the preceding description and shown in the accompanying figures be interpreted as illustrative and not in a limiting sense.

All methods described herein can be performed in any suitable order unless otherwise indicated herein or otherwise clearly contradicted by context. The use of any and all examples, or exemplary language (e.g., “such as”), is intended merely to better illustrate the embodiments described herein and does not pose a limitation on the scope of any embodiments unless otherwise claimed.

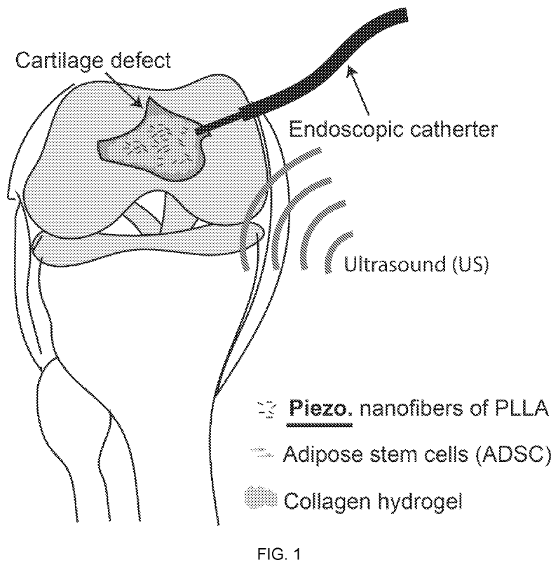

The present disclosure is directed to injectable tissue scaffolds for knee cartilage and many other tissues. FIG. 1 illustrates an injectable piezoelectric hydrogel to treat a cartilage defect in an osteoarthritis knee. The hydrogel scaffold contains short piezoelectric nanofibers of PLLA and adipose-derived stem cells (ADSCs). Under mechanical stimulation (e.g., ultrasound stimulation or joint force), the nanofibers have the ability to generate electricity which then promotes chondrogenic differentiation of encapsulated ADSCs inside the hydrogel or host cells. The piezoelectric injectable hydrogel avoids invasive implantation while still providing the same piezoelectric property to stimulate tissue healing. Tests have shown its effectiveness in vitro. The piezoelectric hydrogel by itself may also receive joint load to produce electrical cues for tissue healing.

### Fabrication of Piezoelectric Nanofibers

Nanofibers of PLLA were electrospun on a drum collector at 4,000 RPM for alignment, stretched and post-processed by annealing at 105° C. and 160° C. obtain highly piezoelectric PLLA nanofibers, according to our previous work and others. The nanofibers are then embedded in an optimum cutting temperature (OCT) gel and cryo-sectioned into short fibers 25 um in length by cryostat. After that, the OCT gel is washed off, and lyophilized to collect the chopped PLLA nanofibers. For the control fiber, PDLLA is used which has no piezoelectricity.

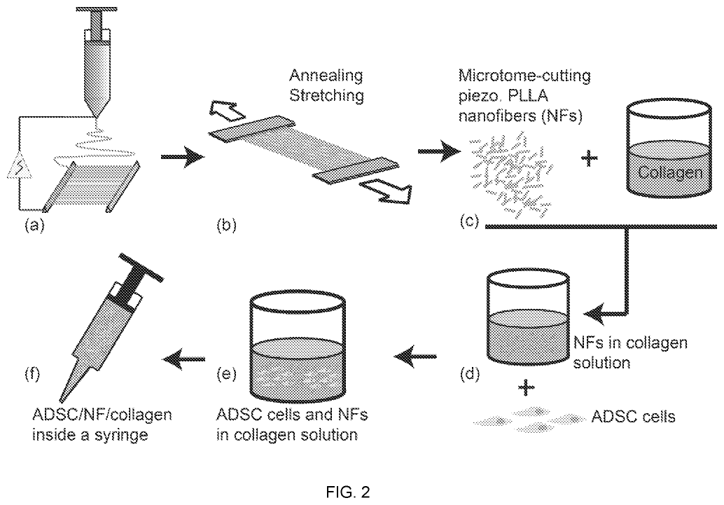

For the in vitro study, we used rabbit adipose derived stem cells (CYAGEN US INC). The ADSC cells from the cryo-reserved stock vials were thawed, and mixed with the collagen hydrogel solution, containing the short PLLA piezo-nanofibers. This homogeneous solution was loaded into a syringe or endoscopic tube for injection, as seen in FIG. 2. For in vivo study, we will use allogenic ADSCs or chondrogenic cells.

As noted above, FIG. 2 illustrates fabrication of the injectable piezoelectric hydrogel. As seen in FIG. 2, the fabrication began with electrospinning of highly aligned PLLA nanofibers (at step (a)). After electrospinning the PLLA nanofibers (at step (a)), annealing and stretching was performed to obtain piezoelectricity from the PLLA nanofibers (at step (b)), following our previous work. At step (c), microtome was used to slice the nanofibers, embedded inside a gel, into short nanofibers, which were mixed with the collagen hydrogel solution, following a published work. At steps (d) and (e), the ADSC cells were mixed with the collagen/nanofiber solution to form a homogenous solution of collage, cells, and piezoelectric nanofibers. At step (f), the final solution was loaded into a syringe.

### Experimental Data

1. Piezoelectricity of PLLA Fibers Under Impact System:

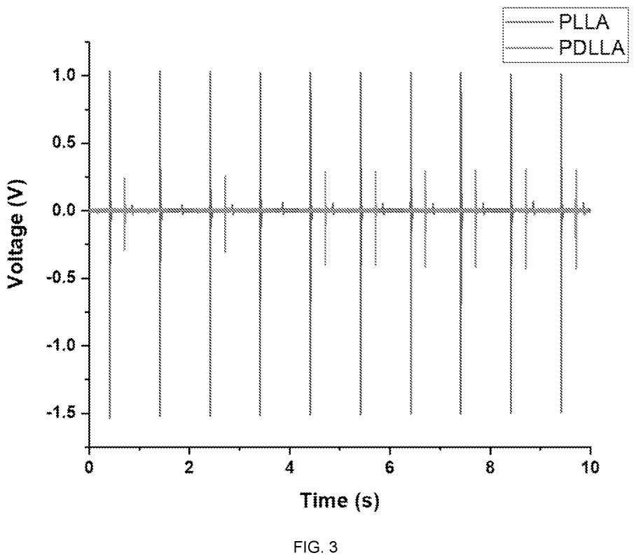

Processed PLLA fibers, which were cut at a 45° angle, with the stretching direction sandwiched between aluminum electrodes, as your previous work. Finally, the electrodes were encapsulated polyimide tape. The PLLA sensor(s) were fixed on a beam in a vibration system and a force was applied to the PLLA sensor(s). Voltage output was plotted as seen in FIG. 3. FIG. 3 illustrates the voltage output of the piezoelectric PLLA fibers versus the non-piezoelectric control fibers. PDLLA was used as control for the experiment. The total voltage generated from the PLLA fibers was around 2.5(V), which is significantly higher than the control fibers.

2. Confirm Piezoelectricity of Cut PLLA Fibers by Rhodamine B Dye (ROS Generation)

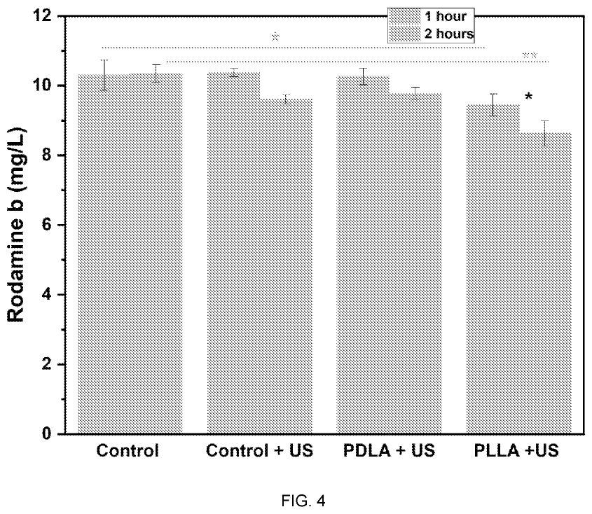

It was reported that when piezoelectric materials vibrate in water, on anode, the piezoelectric materials attract hydrogen and release hydrogen radicals while in cathode, hydroxyl radicals (OH) are generated which can remove dye of molecules. Therefore, to further confirm the piezoelectric property of the chopped off PLLA fibers, we quantify the ability to remove Rhodamine B dye of our fibers. In this experiment, we added 2 mg of either PLLA or PDLLA fibers into 1 ml of Rhodamine B dye solution in 1×PBS pH 7.4 (10 mg/L) and subjected samples to 40 KHz ultrasonication bath for an hour and two hours. The samples with Rhodamine B dye solution alone and Rhodamine B dye solution plus PDLLA fibers served as control groups. As seen in the data plotted in FIG. 4, the dyes were significantly removed in the chopped off PLLA fibers after one hour and two hours (p<0.05 and P<0.01, respectively) compared to other control groups. This proved that after being chopped off, the PLLA fibers still keep their piezoelectric property.

3. Injectable Ability of PLLA Fibers Hydrogel

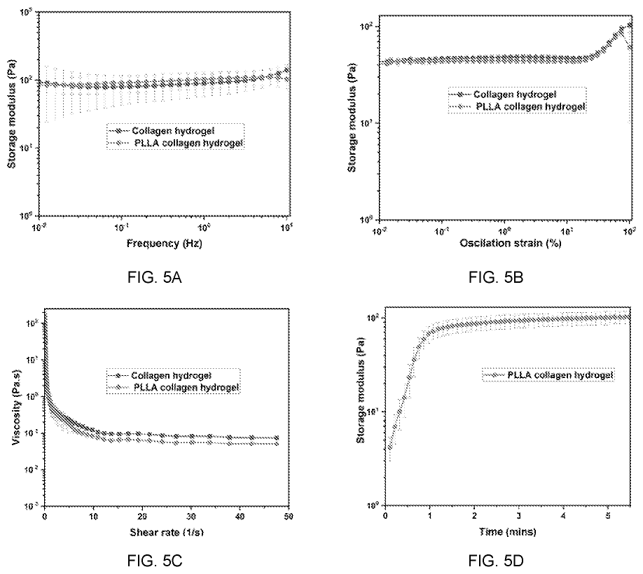

In this experiment, we assessed the injectable property and gelation time of fabricated hydrogel by using rheology measurements. The collagen hydrogel samples were used as control groups. FIGS. 5A-5D are graphs illustrating results of a frequency sweep. FIG. 5B illustrates a strain sweep and FIG. 5C illustrates a continuous flow for Collagen hydrogel and PLLA Collagen hydrogel by rheology measurement at room temperature. FIG. 5D illustrates time gelation of PLLA collagen hydrogel at 37 degrees Celsius. The data in FIGS. 5A-5C illustrate that the viscosity and storage modulus of collagen hydrogel with/without PLLA fibers are similar when subjected to the same range of frequency and oscillation strain. It illustrates that the presence of PLLA fibers in the hydrogel did not affect the injectable property of collagen. FIG. indicates that PLLA collagen hydrogel was solidated after a minute.

4. The Biocompatibility of Piezoelectricity Hydrogel

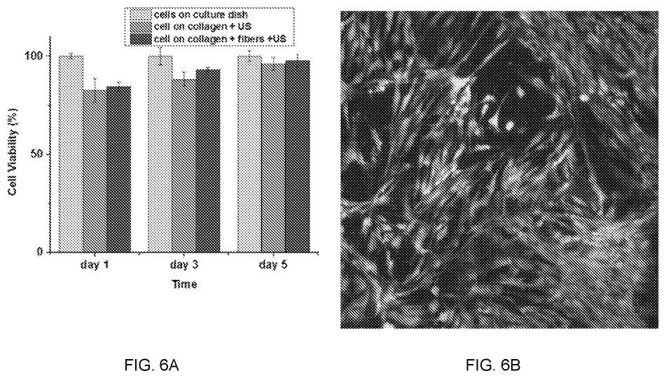

In this experiment, we checked the biocompatibility of fabricated gel by Prestoblue and Cell Live and Dead assay kit. FIGS. 6A-6B illustrate biocompatibility of piezoelectricity hydrogel with ADSC cells. FIG. 6A is a graph of quantitative data at different time points. FIG. 6A illustrates that the cells in fabricated hydrogel and in the same gel, plus ultrasound treatment at different time points, were almost the same to the control group. FIG. 6B is a florescent image of alive ADSC cells (green) and dead ADSC cells (red) in hydrogel at day 5. As indicated in FIG. 6B, most cells were alive and healthy in the hydrogel.

5. The ability to induce chondrogenesis piezoelectricity hydrogel

In this experiment, 10\hat{ }6 ADSC cells were seeded in different hydrogel below with growth media and chondrogenesis media:
Group 1: ADSC+Collagen (control)
Group 2: ADSC+Collagen+PDLLA
Group 3: ADSC+Collagen+PLLA
Group 4: ADSC+Collagen+US
Group 5: ADSC+Collagen+PDLLA+US
Group 6: ADSC+Collagen+PLLA+US

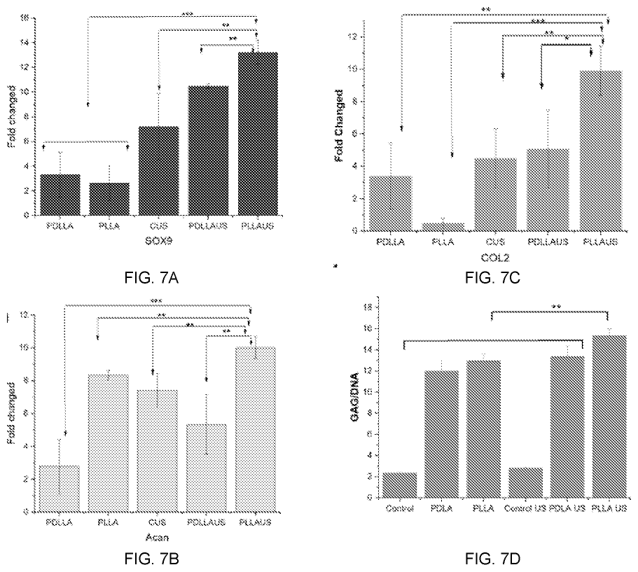

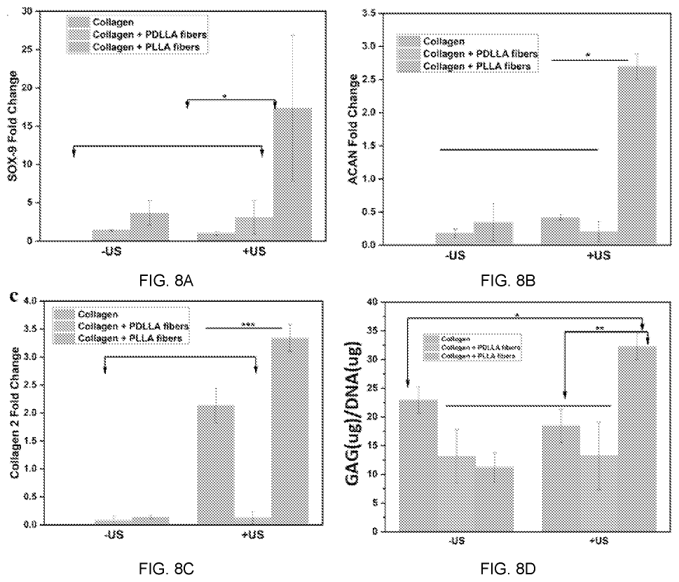

The cells were then kept in a cell incubator at 37° C., 5% CO₂, and 5% O₂ balance with 90% N₂ gas. Groups 4, 5, and 6 received twenty minutes of ultrasound stimulation every day. After 14 days, total RNA was collected and real time qPCR was used to assess SOX9, ACAN, and Collagen II genes expression. B2M was used as the housekeeping gene and data was plotted against the control group. The data showed that Group 6 (piezoelectric hydrogel) had the highest significance in SOX9, ACAN, and Collagen II genes compared to other genes for both growth media (as seen in FIGS. 7A-7D) and chondrogenesis media (as seen in FIGS. 8A-8D). These genes are the markers for cartilage regeneration.

FIGS. 7A-7D illustrate the ability to induce chondrogenesis of fabricated hydrogel in growth media. FIGS. 7A-7C illustrate SOX9, ACAN, and Collagen II gene expression of ADSC's in different groups, respectively. FIG. 7D illustrates the GAG proteins measurement. FIGS. 8A-8D illustrate the ability to promote ADSC cells to differentiate to chondrocyte like cells of fabricated hydrogel in chondrogenesis media. FIGS. 8A-8C illustrate SOX9, ACAN, and Collagen II gene expression of ADSCs in different groups, respectively. FIG. 8D illustrates the GAG proteins measurement.

6. Piezoelectricity of PLLA Fibers Under 40 KHz Ultrasound Stimulation in Rabbit's Knee Cadaver Defect

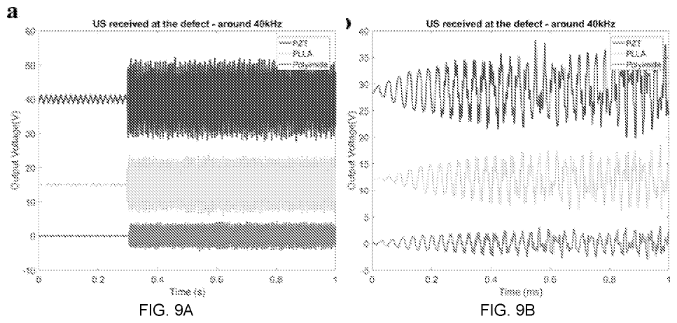

Before starting the pilot study, we wanted to confirm that when stimulating piezoelectric hydrogel with ultrasound, the signal could penetrate the thick layer of ligament in the rabbit's knee and reach PLLA collagen hydrogel in the defect so that the fabricated hydrogel could produce electrical charges. To do that, we created an osteochondral defect of ˜4 mm in diameter and 2 mm in depth. Three sensors made of different materials including PZT sensor for positive piezoelectric control group, polyimide negative non-piezoelectric control group, and PLLA for experimental group. FIGS. 9A-9B illustrate the voltage output of the three sensors, where FIG. 9B is a zoomed in version of the voltage output of FIG. 9A. FIG. 9A illustrates that ultrasound stimulation penetrated through the alignment layer and reached the sensors. The PZT and PLLA sensors generated around 15V and 10V, respectively, while non-piezoelectric polyimide just showed noise.

Various features and advantages of certain embodiments are set forth in the following claims.

What is claimed is:

1. A method for osteoarthritis treatment, the method comprising:
providing a cartilage hydrogel, the cartilage hydrogel including piezoelectric nano-fibers of Poly-L-lactide (PLLA);
injecting the cartilage hydrogel into a cartilage defect;
applying an ultrasonic treatment to the cartilage defect; and
in response to applying the ultrasonic treatment to the cartilage defect,
converting a mechanical impact of the ultrasonic treatment into an electrical charge from the piezoelectric nano-fibers of PLLA, and
providing, in response to the electrical charge from the piezoelectric nano-fibers of PLLA, chondrogenesis differentiation for cartilage regeneration for the cartilage defect.

2. The method of claim 1, wherein applying the ultrasonic treatment to the cartilage defect includes directly applying the ultrasonic treatment to the cartilage defect.

3. The method of claim 1, wherein providing the cartilage hydrogel includes providing a cartilage hydrogel that includes the piezoelectric nano-fibers of PLLA and a collagen hydrogel.

4. The method of claim 1, wherein providing the cartilage hydrogel includes providing a cartilage hydrogel that includes the piezoelectric nano-fibers of PLLA and a collagen hydrogel with adipose-derived stem cells.

5. The method of claim 1, wherein providing the cartilage hydrogel includes providing a cartilage hydrogel that is biocompatible and biodegradable.

6. The method of claim 1, further comprising:
fabricating the cartilage hydrogel.

7. The method of claim 6, wherein fabricating the cartilage hydrogel includes electrospinning aligned PLLA nano-fibers.

8. The method of claim 7, wherein fabricating the cartilage hydrogel includes generating a piezoelectric property of the piezoelectric nano-fibers of PLLA by annealing and stretching nano-fibers of PLLA after electrospinning the aligned PLLA nano-fibers.

9. The method of claim 1, wherein injecting the cartilage hydrogel into the cartilage defect includes injecting the cartilage hydrogel into the cartilage defect via an arthroscopic procedure.

10. A system for osteoarthritis treatment, the system comprising:
a cartilage hydrogel, the cartilage hydrogel including piezoelectric nano-fibers of Poly-L-lactide (PLLA), wherein the cartilage hydrogel is injected into a cartilage defect, and wherein the cartilage hydrogel is configured to receive an ultrasonic treatment at the cartilage defect, and
wherein, in response to the ultrasonic treatment, the piezoelectric nano-fibers of PLLA converts a mechanical impact of the ultrasonic treatment into an electrical charge, wherein the electrical charge triggers a chondrogenesis differentiation for cartilage regeneration for the cartilage defect.

11. The system of claim 10, wherein the ultrasonic treatment is received directly by the cartilage defect.

12. The system of claim 10, wherein the cartilage hydrogel includes the piezoelectric nano-fibers of PLLA and a collagen hydrogel.

13. The system of claim 10, wherein the cartilage hydrogel includes the piezoelectric nano-fibers of PLLA and a collagen hydrogel with adipose-derived stem cells.

14. The system of claim 10, wherein the cartilage hydrogel is biocompatible and biodegradable.

15. The system of claim 10, wherein the cartilage hydrogel is fabricated by electrospinning aligned PLLA nano-fibers.

16. The system of claim 15, wherein the cartilage hydrogel is further fabricated by
generating a piezoelectric property of the piezoelectric nano-fibers of PLLA by annealing and stretching nano-fibers of PLLA after electrospinning the aligned PLLA nano-fibers.

17. The system of claim 10, wherein the cartilage hydrogel is injected into the cartilage defect via an arthroscopic procedure.

18. The system of claim 10, wherein the electrical charge is a surface charge that promotes the chondrogenesis differentiation.
